(19) United States
(12) Patent Application Publication
(10) Pub. No.: US 2020/0179539 A1
(43) Pub. Date: Jun. 11, 2020

(54) Contrast Agents and Methods of Making the Same for Spectral CT That Exhibit Cloaking and Auto-Segmentation

(71) Applicant: BOARD OF REGENTS, THE UNIVERSITY OF TEXAS SYSTEM

(72) Inventors: Matthew A. Lewis, Farmers Branch, TX (US); Khaled A. Nasr, Dallas, TX (US); Todd C. Soesbe, Dallas, TX (US); Robert E. Lenkinski, Dallas, TX (US)

(21) Appl. No.: 16/311,105

(22) PCT Filed: Jun. 22, 2017
(86) PCT No.: PCT/US2017/038802

§ 371 Date: Dec. 18, 2018

(51) Int. Cl. A61K 49/04; A61K 9/00

(52) U.S. Cl. CPC A61K 49/0428; A61K 9/0019

(57) ABSTRACT

The present invention includes a composition, method, method of making, and a kit for using an enteric contrast agent formulation comprising an enteric contrast medium comprising particles comprising atoms of an element with an atomic number from 70 to 77, and a pharmaceutically acceptable vehicle in which the particles are dispersed.

### TECHNICAL FIELD OF THE INVENTION

The present invention relates in general to the field of contrast agents, and more particularly, to a new class of contrast agents for Spectral CT that exhibit cloaking and auto-segmentation.

### BACKGROUND OF THE INVENTION

Without limiting the scope of the invention, its background is described in connection with Spectral CT and contrast agents.

Recent developments in clinical spectral computed tomography (Spectral CT) scanners have enabled rapid and simultaneous acquisitions of in vivo images [1, 2]. Spectral scanners are either two separate X-ray source (dual energy) or a single source with dual detectors that provide material decomposition images of clinical contrast agents [3]. In vivo separation of two different contrast agents administered simultaneously has been reported for dual-energy source CT scanners [4-6]. Dual-energy material separation is determined by the ratios of the X-ray attenuation coefficients between high and low energies. The attenuation ratio method is limited to qualitative rather than quantitative evaluations of the contrast media and no clear separation was observed. Multispectral CT would benefit from a new generation of contrast agents.

### SUMMARY OF THE INVENTION

In one embodiment, the present invention includes an enteric contrast agent formulation comprising: an enteric contrast medium comprising particles comprising atoms of an element with an atomic number from 70 to 79, and in certain embodiments 70 to 77; and a pharmaceutically acceptable vehicle in which the particles are dispersed. In one aspect, the element is invisible or cloaked in an iodine spectral computer tomography image. In another aspect, the element is in a compound selected from Rhenium(VII) sulfide (Re₂S₇), or a non-soluble Tungstate (X—WO₄). In another aspect, the element is non-absorbable in the gut. In another aspect, the element is non-radioactive. In another aspect, the element is selected from Yb, Lu, Hf, Ta, W, Re, Os, Au, or Ir. In another aspect, the element has a Z<83 (Bismuth). In another aspect, the particles are coated with a viscosity modifier and water retention agent to form a colloidal nanoparticle that is pseudo-cloaking. In another aspect, the particles are provided in an enteric coating. In another aspect, the particles are adapted for oral administration. In another aspect, the atoms are defined further as comprising particles of a material selected from microparticles and nanoparticles, wherein the particles comprise atoms of an element with an atomic number from 70 to 79, or 70 to 77. In another aspect, the enteric contrast medium is defined further as comprising a first subpopulation of atoms having a first atomic number and a second subpopulation of atoms having a second atomic number, wherein the first atomic number and the second atomic number are different atomic numbers. In another aspect, the particle is coated with a material compatible with enteric administration of the formulation. In another aspect, the particle is essentially water insoluble or slightly water-soluble. In another aspect, the element further comprises one or more atoms selected from oxygen and sulfur forming a compound with the element. In another aspect, the element is in the form of an oxide, a carbonate, a borate, a hydroxide, a phosphate, and a salt of an organic acid of the element. In another aspect, the formulation is in a form selected from a suspension, a colloid, an emulsion, a hydrogel, or a combination thereof. In another aspect, about 10% (w/w) to about 90% (w/w) of the weight of the formulation is the contrast material particles, or about 30% (w/w) to about 70% (w/w) of the weight of the formulation is the contrast material particles. In another aspect, the coating comprises a water-soluble polymer. In another aspect, the coating comprises a polymer which is a member selected from a poly(alkylene oxide), a poly(amino acid), a poly(ester) polymer, a polysaccharide, polyvinylpyrrolidone, a polyvinyl) polymer, a poly(ethylene imine) polymer, a poly(acrylic) polymer, a poly(siloxane) polymer, a protein, a dendrimer and a combination thereof. In another aspect, the coating comprises an organic molecule with a molecular weight of less than about 3,000 Daltons. In another aspect, the coating comprises an organic molecule with a molecular weight of less than about 3,000 Daltons, which is a member selected from an organic acid, alcohol, amine, an oligosaccharide and their derivatives and analogs (e.g. perfluoroalkyl chain, fluoroalkyl chain) and a combination thereof. In another aspect, the size of the particles is from: about 1 nm to about 500 microns, less than about 200 nm, from about 200 nm to about 5 microns, about 1 micron to about 50 microns, or greater than about 50 microns. In another aspect, the pharmaceutically acceptable vehicle comprises an aqueous medium, further comprising an additive to retard dehydration of the formulation in the bowel, a flavoring agent, a thickening agent, a suspending agent, a flow agent, a pH buffer and a combination thereof.

Another embodiment of the present invention includes a method of making a colloidal nanoparticle contrast agent for enhanced spectral CT image of a subject comprising: suspending a nanopowder comprising an element selected from Z=70 (Ytterbium) to Z=78 (Platinum) or even Z=79 (Gold) in water; and coating the nanopowder with a sufficient amount of a viscosity modifier and water retention agent to form a colloidal nanoparticle that is pseudo-cloaking. In one aspect, the method further comprises the step of adding one or more additives, stabilizers, adhesives, flavorants, or preservatives. In another aspect, the method further comprises the step of adding one or more additives, stabilizers, adhesives, flavorants, or preservatives selected from at least one of: bentonite, dimethylpolysiloxane 200, dimethylpolysiloxane 1000, D-sorbitol, D-mannitol, saccharin sodium salt hydrate, sodium benzoate, or sodium citrate. In another aspect, the element has a Z<83 (Bismuth). In one aspect, the nanopowder is selected from at least one of Yb, Lu, Hf, Ta, W, Re, Os, Ir, Au, tantalum oxide, tungsten carbide, tungsten trioxide, sodium tungstate, or rhenium sulfide. In another aspect, the step of coating the nanoparticle comprises at least one of sonicating or stirred for an amount of time sufficient to obtain the colloidal nanoparticles. In another aspect, the step of coating the nanoparticle comprises at least one of sonicating or stirred for an amount of time sufficient to obtain homogenous colloidal nanoparticles.

Yet another embodiment of the present invention includes a method of acquiring a contrast enhanced spectral CT image of a subject comprising: administering to the subject a diagnostically effective amount of an enteric contrast medium formulation comprising particles of an element having an atomic number from 70 to 79, in some cases Z=70 to Z=77, dispersed in a pharmaceutically acceptable vehicle; and acquiring the image of the subject. In one aspect, the X-ray image is a computed tomography image. In another aspect, the image is an image of a region selected from the abdomen and pelvis of the subject. In another aspect, the element has a Z<83 (Bismuth). In another aspect, the method further comprises administering to the subject a second contrast medium different from the enteric contrast medium, and the second contrast medium is administered through a route selected from oral administration, intrathecal administration, intravesicular administration, enteric administration, anal administration and intravascular administration. In another aspect, the enteric contrast medium and the second contrast medium are distinguishable from each other in the image. In another aspect, the enteric contrast medium does not appear in an IodineNoWater image. In another aspect, the enteric contrast medium remains in the WaterNoIodine image and is enhanced when compared to a conventional CT image. In another aspect, an image of a bowel can be segment by performing a pixel wise comparison of the WaterNoIodine and conventional CT images. In another aspect, the particles are coated with a viscosity modifier and water retention agent to form a colloidal nanoparticle that is pseudo-cloaking. In another aspect, the enteric contrast agent is administered to the subject by delivery through: a natural cavity selected from the mouth, vagina, bladder, rectum and urethra; a surgically created space selected from an ileal pouch, and a neobladder; or medical device selected from a catheter, a tube, a reservoir, a pouch and a pump.

Yet another aspect of the present invention includes a kit comprising: a first vial containing the enteric contrast medium a diagnostically effective amount of the enteric contrast medium formulation comprising an atomic number from 70 to 79, and in some aspected Z=70 to Z=77 dispersed in a pharmaceutically acceptable vehicle; and directions for formulating the enteric contrast medium, the second contrast medium or a combination thereof. In one aspect, the kit further comprises a second vial containing a second contrast medium for intravenous administration. In one aspect, the element has a Z<83 (Bismuth).

### BRIEF DESCRIPTION OF THE DRAWINGS

For a more complete understanding of the features and advantages of the present invention, reference is now made to the detailed description of the invention along with the accompanying figures and in which:

FIG. 1 shows a schematic representing the spectral CT two-material decomposition algorithm for water and iodine. The conventional CT image (top) of the total attenuation (μₜₒₜₐₗ) is divided into two images (below), where one represents the attenuation due to water equivalent materials (μ_{water}) and the other the attenuation due to iodine equivalent materials (μ_{iodine}) Materials that are neither water nor iodine typically appear in both images (e.g., the calcium in bone or the metal in the stent) or in only one image (e.g., the barium in the bowel). These axial images are from a 71 year old male who received both oral contrast (i.e., barium) and intravenous contrast (i.e, iodine).

FIG. 2 is a plot of mass attenuation coefficients versus diagnostic x-ray energy for iodine (red), tungsten (green), and bismuth (blue)(9). The vertical dashed lines represent typical average x-ray energies for the low (57 keV) and high (83 keV) x-ray spectra associated with dual-energy spectral CT. The vertical discontinuity in each colored line represents the increase in attenuation due to the K-edge absorption of that element. The K-edge energy for each element is shown above the corresponding increase in attenuation.

FIG. 3 is a periodic table showing the range of pseudo-cloaking elements (grayed) for the detector-based Philips IQon spectral CT system for use with the method of the present invention. These data were simulated using the custom MATLAB computer program. These pseudo-cloaking high-Z elements (ytterbium through platinum) will have ≤0 mg I/mL pixel values when viewed in the iodine equivalent image (i.e., the iodine map).

FIGS. 4A to 4D show pictures of six 50 mL plastic vials that contain the five contrast agents used in this study along with a vial of pure water. FIG. 4A shows a picture of 50 mL plastic vials containing the contrast agents used in this study. In FIG. 4B the conventional, and in FIG. 4C water equivalent, and FIG. 4D iodine equivalent 3 mm thick axial images were produced from a Philips IQon detector-based spectral CT scanner. The average pixel value is shown below each vial.

FIGS. 5A to 5D show axial images of the rat that was administered intravenous iodine and oral barium simultaneously. Each image is the same axial slice displayed using a different spectral CT method. FIG. 5A shows a conventional image showing iodine in the kidneys and barium in the bowel lumen. FIG. 5B shows the water equivalent image where both iodine and barium have been removed. FIG. 5C shows the iodine equivalent image where both iodine and barium appear. FIG. 5D shows a colorized iodine equivalent image overlaid on top of the conventional image emphasizing that the iodine and barium are not differentiated in the bowel.

FIGS. 6A to 6D show axial images of the rat that was administered intravenous iodine and oral tungsten simultaneously. Each image is the same axial slice displayed using a different spectral CT method. FIG. 6A shows the conventional image showing iodine in the kidneys and tungsten in the bowel lumen. FIG. 6B shows the water equivalent image where only the iodine has been removed. FIG. 6C shows the iodine equivalent image where the iodine appears and the tungsten is pseudo-cloaked. FIG. 6D shows the colorized iodine equivalent image overlaid on top of the conventional image where the iodine in the bowel wall (green pixels) can now be differentiated from the tungsten in the bowel lumen.

FIGS. 7A to 7D show axial images of the rat that was administered intravenous iodine and oral rhenium simultaneously. Each image is the same axial slice displayed using a different spectral CT method. FIG. 7A shows the conventional image showing iodine in the kidneys and rhenium in the bowel lumen. FIG. 7C shows the water equivalent image where only the iodine has been removed. FIG. 7C shows the iodine equivalent image where the iodine appears and the rhenium is pseudo-cloaked. FIG. 7D shows the colorized iodine equivalent image overlaid on top of the conventional image where the iodine in the bowel wall (green pixels) can now be differentiated from the rhenium in the bowel lumen.

FIGS. 8A-8D show post-contrast CT images of a 71-year-old male patient with a large abdominal aneurysm in accordance with the present invention. FIG. 8A shows the conventional image showing the metallic stents and iodine within the aortic bifurcation (arrows) and the tantalum within the Onyx embolic agent (arrows). FIG. 8B shows the water equivalent image where only the iodine has been removed. FIG. 8C shows the 200 keV mono-energetic image showing little reduction in the streak artifacts caused by the tantalum. FIG. 8D shows the iodine equivalent image where the tantalum and artifacts are pseudo-cloaked reveling a suspect type 2 endoleak (arrow).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention includes a novel class of contrast agents that can be ‘cloaked’ or made invisible on certain images produced by various spectral CT scanners, including systems by Siemens and Philips. It was found that spectral CT scanner produce images with entirely different image formation chains, but the class of agents used herein demonstrates the same behavior in both systems. By way of explanation the mathematical nature of the cloaking ability has been determined.

The present invention includes contrast agents containing certain elements, ranging from ytterbium (₇₀Yb) to iridium (₇₇Ir) on the periodic table, have a peculiar and unexpected property when viewed on Spectral CT using an Iodine-Water decomposition that have been adapted for enteral use. Normally, highly attenuating materials such as barium, bismuth, or calcium in bone will show up in the IodineNoWater image. However, for certain Iodine-Water decomposition image formation chains, highly attenuating elements in the Yb—Ir range will be invisible, or cloaked, in the Iodine image. The present invention was tested with toySDCT simulations in a Philips IQon, a Siemens Force, and a Siemens Flash, however, any equivalent device and software can be used with the present invention.

In some imaging scenarios, Iodine and the cloaking element may be in separate compartments. For example, consider an iodine contrast study where the patient was also given oral contrast akin to barium sulfate suspension but containing instead an element in the Yb—Ir range. Normally, barium contrast in the bowel shows up in the IodineNoWater image because the iodine and barium are very similar in attenuation. However, using these special elements, the bowel contrast will not appear in the IodineNoWater image. Furthermore, a second remarkable property comes into play here: the bowel contrast will not only remain in the WaterNoIodine image, it with be enhanced compared to the conventional CT image. Therefore, to quickly segment the bowel, one only needs to perform a pixel wise comparison of the WaterNoIodine and conventional CT images (WaterNoIodine>conventional gives mask for bowel contrast). The present invention was tested with toySDCT simulations in a Philips IQon, a Siemens Force, and a Siemens Flash.

Considering the opposite case, the iodine and cloaking element may be in same compartment. For example, consider IV iodine contrast and a salt such as potassium perrhenate (containing rhenium) in the vasculature. If sufficient amounts of the cloaking element are present in the compartment, then no iodine contrast will appear in the IodineNoWater. The iodine contrast will be effectively cloaked. For insufficient amounts of cloaking element, the iodine will appear in the IodineNoWater image, but it will be underestimated. The present invention was tested with toySDCT simulations in a Philips IQon spectral CT scanner.

Considering a tissue with a known concentration of the cloaking agent (e.g., using MRI methods), then the sensitivity floor for iodine contrast imaging will be increased. Thus, the critical concentration of iodine contrast can then be detected using a zero crossing algorithm.

Composition and Preparation of High-Z Elements Colloidal Nanoparticles as Oral X-ray Contrast Media that exhibit Pseudo-cloaking and Auto-segmentation (PCCM) in spectral CT.

The present inventors made and used the novel contrast agents described herein, namely, the pseudo-cloaking properties of high-Z elements (Z=70 (Ytterbium) to Z=78 (Platinum)), in detection-based spectral CT. A clear separation was observed between pseudo-cloaking contrast media (PCCM's) of high-Z elements (Ytterbium (Z=70) to platinum (Z=78)) or even gold (Z=79) and iodine-based contrast media in both ex vivo and in vivo using clinical detection-based spectral CT. It has been found that pseudo-cloaking range can extend up to gold (Z=79) or more, but definitely ends before Bismuth (Z=83). Two contrast media, one made from high-Z elements and the second made from iodine-based or barium-based contrast media could be used simultaneously to distinguish between an oral contrast and vascular contrast in a single CT examination. Contrast agents with Ytterbium[8, 9], Tantalum[10-12], Rhenium, Tungsten[13] [14, 15] and Platinum[16] were reported and used as a conventional CT contrast agents. Unfortunately, these high-Z elements have unknown or high toxicity (LD₅₀) making them unsuitable to be used for in vivo CT imaging.

In one example, the present invention provides a novel pseudo-cloaking contrast media (PCCM) for in vivo applications as an oral contrast media. The invention provides the composition of colloidal nanoparticle of compounds of elements with Z=70 to Z=78. The invention includes a novel PCCM suspension which will be stable, palatable, compatible with stomach fluids and which will provide a smooth, even, long-lasting coating on the lining of the stomach, small bowel and colon for CT applications.

A typical colloidal nanoparticle (20 mg/mL of desired element Z=70 to Z=78 in 2% CMC) within the scope of this invention is prepared as follows:

A selected nanopowder of elements with Z=70 (Ytterbium) to Z=78 (Platinum) was suspended in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

From compounds of high-Z elements Z=70 to Z=78, the inventors selected tantalum (LD₅₀=2,500 mg/Kg rat oral), tantalum oxide (LD₅₀=8,000 mg/Kg rat oral), tungsten (LD₅₀=5,000 mg/Kg rat dermal), tungsten carbide (LD₅₀>2,000 mg/Kg rat oral, >2,000 mg/kg rat dermal), tungsten trioxide (LD₅₀=1059 mg/Kg rat oral) sodium tungstate (LD₅₀=1,190 mg/Kg rat oral), and rhenium sulfide (LD₅₀=2,800 mg/Kg rat oral) as low toxicity compounds of high-Z elements that exhibit pseudo-cloaking.

### Example 1: Synthesis of Tantalum Colloidal (20 mg/mL Ta)

Tantalum powder (60-100 nm, 2 g) was suspended in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

### Example 2: Synthesis of Tantalum Oxide Colloidal (20 mg/mL Ta)

Tantalum oxide (4.9 g) was suspended in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

### Example 3: Synthesis of Tungsten Colloidal (20 mg/mL W)

Tungsten (2 g) was suspended in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

### Example 4: Synthesis of Tungsten Carbide Colloidal (20 mg/mL W)

Tungsten carbide (2.13 g) was suspended in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

### Example 5: Synthesis of Tungsten Oxide Colloidal (20 mg/mL W)

Tungsten oxide (2.52 g) was suspended in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

### Example 6: Synthesis of Sodium Tungstate Colloidal (20 mg/mL W)

Sodium tungstate (3.58 g) was dissolved in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

### Example 7: Synthesis of Rhenium Sulfide (20 mg/mL Re)

Rhenium sulfide (2.68 g) was suspended in 100 mL distilled water. Carboxymethyl cellulose sodium salt (2 g), bentonite (0.1 g), dimethylpolysiloxane 200 (0.1 g), dimethylpolysiloxane 1000 (0.1 g), D-sorbitol (0.05 g), D-mannitol (0.05 g), saccharin sodium salt hydrate (0.05 g), sodium benzoate (0.02 g), sodium citrate (0.02 g) were added to the 2% aqueous solution of carboxymethyl cellulose. The resulting suspension was sonicated for 30 minutes and stirred for 1 hour in order to obtain a homogenous colloidal nanoparticle.

The colloidal nanoparticles of selected compounds were evaluated for ex vivo and in vivo imaging. Colloidal nanoparticles of low toxicity compounds of tantalum, tungsten and rhenium were shown to be excellent candidates of PCCM's providing a clear separation from iodine-based contrast media observed in phantom and in vivo imaging using detection-based spectral CT (IQon, Philips Healthcare). Compounds with high-Z elements (Z=70 to Z=78) are shown to be ideal for future development of contrast agents that exhibit the pseudo-cloaking phenomenon. Thus, high-Z element PCCM's provide clear oral and vascular differentiation in a single CT examination detection-based spectral CT (IQon, Philips Healthcare).

### Example 8. The Separation of Simultaneously Administered Intravascular and Oral X-Ray Contrast Agents Using Spectral CT: Examples of Pseudo-Cloaking with High-Z Materials

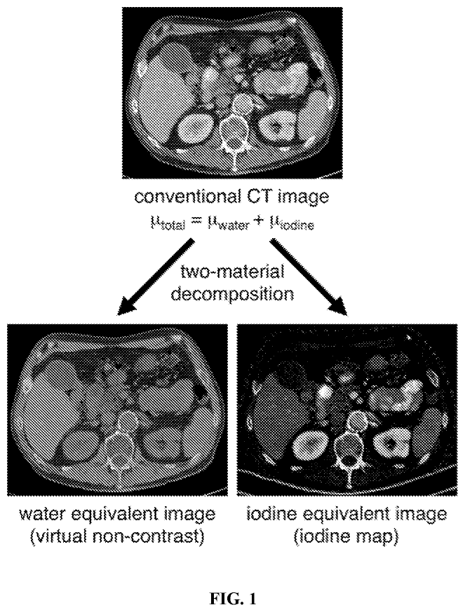

Over the past decade one of the most frequently used spectral CT protocols has been the two-material decomposition that uses water and iodine as the basis pair (1-3). In this protocol, the total x-ray beam attenuation was separated into two components, with one component representing the attenuation due to “water equivalent” materials and the other representing the attenuation due to “iodine equivalent” materials. This allows the conventional CT image to be separated into two spectral CT images, with one showing the attenuation due to water equivalent materials (expressed in Hounsfield units) and the other showing the attenuation due to iodine equivalent materials (usually expressed in mg of iodine per mL) as shown in FIG. 1. This protocol has found several useful applications in diagnostic spectral CT including using virtual non-contrast images to reduce radiation dose and establishing iodine biomarkers for improved renal lesion diagnosis (4,5).

Although the two-material decomposition algorithm works well for separating the low-Z water elements (Z=1 and 8) from the high-Z iodine (Z=53) there are undoubtedly other elements besides water and iodine found in vivo. Imaging these other elements with a two-material decomposition based on, and designed for, water and iodine lead to some interesting results. For example, calcium (Z=20) has a relatively low atomic number when compared to iodine, yet in solid bone mineral (hydroxyapatite) it is highly attenuating. As a result, the water and iodine two-material decomposition algorithm puts some of the calcium attenuation into the water equivalent image and the rest into the iodine equivalent image. This can be observed by noting that bone appears in both the water equivalent image and the iodine equivalent image in FIG. 1. Barium (Z=56), which is currently the only other FDA approved CT contrast agent besides iodine, has an atomic number that is just three units higher than iodine and thus they share very similar mass attenuation coefficients (6). The attenuation due to barium is therefore placed entirely into the iodine equivalent image as observed by the barium-based oral contrast seen in FIG. 1. Bismuth (Z=83), which has been proposed as new CT contrast agent (7), has both high K-edge energy and high attenuation yet is split between the water and iodine images in a manner similar to calcium (8). From these observations the skilled artisan would have expected that, if an element's atomic number is close to water or iodine, then it will be placed into that corresponding image, otherwise it will be split between the two spectral CT images.

While exploring the behavior of exogenous elements under the water and iodine two-material decomposition the present inventors observed that the attenuation from a certain range of high-Z elements is, in a counterintuitive and surprising manner, placed entirely into the water equivalent image. The approximate range of these elements is from ytterbium (Z=70) to platinum (Z=78) and depends slightly on what type of dual-energy spectral CT system is used (i.e., dual-source, fast kVp switching, or detector-based). The inventors define this surprising and unexpected property of the two-material decomposition algorithm as “pseudo-cloaking” because the high-Z elements seem to disappear in the iodine equivalent images. By way of explanation, and in no way a limitation of the present invention, the inventors hypothesized that pseudo-cloaking could be used to visually segment iodine from these high-Z elements. The purpose of this example was to demonstrate that pseudo-cloaking of high-Z elements can be used to segment iodine-based intravascular contrast agents from tantalum, tungsten, and rhenium-based oral contrast agents that were administered simultaneously in an animal model.

Simulations. Computer simulations were performed using a customized CT simulator written in MATLAB software (MathWorks) in order to better understand the phenomenon of pseudo-cloaking. This simulator used the x-ray tube energy spectrum S(E), the spectral attenuation coefficients of the object r) (9), the detector responsivity D(E), and the MATLAB Radon transform function to generate the high and low kVp sinograms for a single two-dimensional axial slice (10). The high and low kVp sinograms were then used as the inputs for either projection-based (11,12) or image-based (13,14) dual-energy spectral CT reconstruction algorithms. In this manner, the water and iodine two-material decomposition images from a variety of different high-Z elements could be simulated for specific dual-energy spectral CT systems (i.e., dual-source, fast kVp switching, and detector-based).

Contrast Agents. These studies used the simultaneous administration of a single intravenous contrast agent and a single oral contrast agent. The intravenous contrast was based on iodine (I) while the oral contrast was based on barium (Ba), tantalum (Ta), tungsten (W), or rhenium (Re). FDA approved Isovue-370 (Bracco Diagnostics, 370 mg I/mL) was used for all intravenous contrast. Also, FDA approved barium sulfate (Bracco Diagnostics, 12 mg Ba/mL) was used for the barium oral contrast. The tantalum, tungsten, and rhenium oral contrast agents were created from tantalum oxide (TaO, 20 mg Ta/mL), tungsten carbide (WC, 20 mg W/mL), and rhenium sulfide (ReS₂, 20 mg Re/mL) nanopowder colloidal suspensions in methylcellulose. Further details describing the chemical synthesis of the tantalum, tungsten, and rhenium oral contrast agents can be found in the Supplemental Material.

Preclinical Protocol. The following protocol was approved by the institutional animal care and use committee (IACUC). Four female Fischer were fasted for 24 hours to clear the gastrointestinal tracts. The rats were approximately seven weeks old and had an average mass of 150 grams. After fasting, the rats were each given a 2 mL dose of oral contrast that was administered using a 15 gauge×100 mm plastic feeding tube (Instech Laboratories) and a 3 mL syringe. The oral contrast was based on barium (Z=56), tantalum (Z=73), tungsten (Z=74), or rhenium (Z=75), with only one oral contrast type given to each animal. Fasting was continued for thirty minutes after the first oral contrast dose after which the animals were given a second 2 mL dose of the same oral contrast. In order to minimize stress and improve procedure success the animals were slightly anesthetized with 2% isoflurane gas just before each oral gavage. After the second dose of oral contrast the animals were put under 2% isoflurane gas and given a 0.3 mL dose of Isovue-370 (Z=53, 370 mg I/mL) that was administered via tail vein injection using a 30 gauge needle and 1 mL syringe. Immediately following the intravenous injection the anesthetized rats were euthanized using carbon dioxide gas then transferred to the detector-based IQon spectral CT scanner (Philips Healthcare) for imaging. The cadaveric animals were scanned individually in the prone position. This method resulted in oral contrast being present in the stomach and bowel lumen and intravenous contrast being present in the kidneys (nephrographic or late phase) and bowel wall.

Preclinical Scan Parameters. The cadaveric rats were scanned on a Philips IQon spectral CT system at 120 kVp, 16×0.625 collimation, and 150 mAs using a QA Body Axial 2D protocol. A field of view of 100 mm and a total length of 200 mm were used, with data being acquired in axial mode (i.e., step and shoot). This produced an in-plane resolution of 0.2 mm per pixel in the spectrally derived images (512×512 pixels) and 320 axial slices (0.625 mm slice thickness). This geometry allowed for high-resolution full body scanning of each rat. The C (i.e., sharp) filter was used during image reconstruction to help improve the spatial resolution. All images were analyzed using the thin-client Spectral Diagnostic Suite software (SpDS, Philips Healthcare).

Clinical Use. Although tantalum, tungsten, and rhenium are currently not approved by the FDA for oral contrast, there is an FDA approved drug that does contain tantalum. The Onyx liquid embolic agent (eV3, Plymouth, Minn.) is used to perform endovascular embolization of aneurysms and contains a high amount of micronized tantalum powder (˜100 mg Ta/mL) in order to provide contrast during fluoroscopy. This Onyx agent was observed in a patient who was scanned with a detector-based IQon spectral CT system under a separate IRB approved research protocol not directly associated with this study. This patient was scanned using a CTA Aorta protocol at 120 kVp using a field of view of 435 mm and a slice thickness of 2 mm. The water and iodine two-material decomposition images from this research patient are included here to demonstrate the feasibility of using pseudo-cloaking in humans.

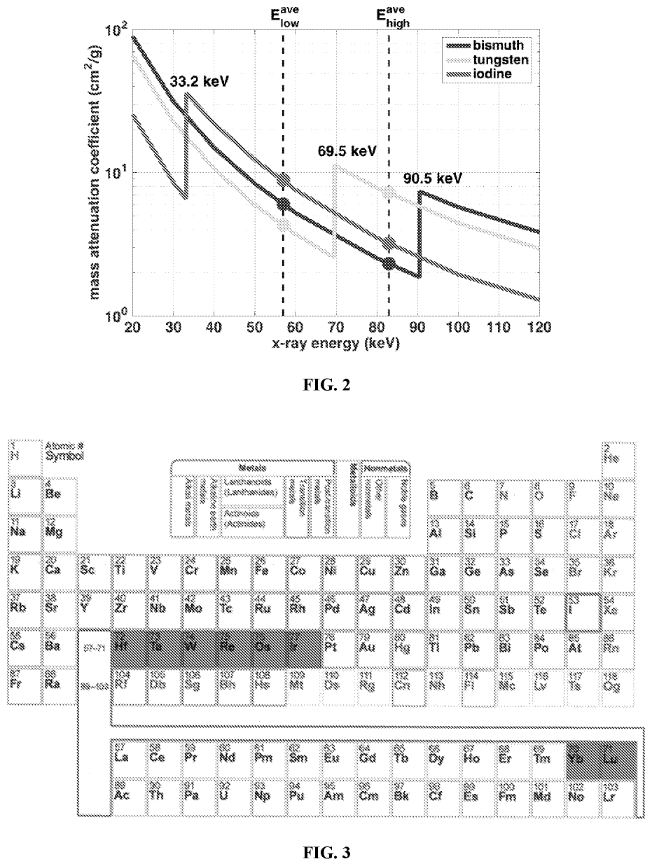

Simulation Results. The computer simulations revealed that pseudo-cloaking is based on a K-edge phenomena that occurs in dual-energy spectral CT systems and is explained as follows. The high and low kVp x-ray spectra that are used in dual-energy spectral CT are associated with a high and low average keV x-ray energy, respectively. Typically only 15 to 30 keV separate the two average energies, yet this provides adequate attenuation differences between the two kVp settings for spectral CT analysis. FIG. 2 shows a plot of the mass attenuation coefficients for iodine, tungsten, and bismuth versus typical diagnostic x-ray energies (9). Also shown in FIG. 2 are two vertical dashed lines that represent the low and high kVp average energies of 57 and 83 keV, respectively. FIG. 2 shows that if the K-edge energy of an element is below the low kVp average energy (e.g., iodine at 33.2 keV) then the attenuation at the low average energy is greater than the attenuation at the high average energy as shown by the two red circular markers. Likewise, the same is true when the K-edge energy is above the high kVp average energy (e.g., bismuth at 90.5 keV) as shown by the two blue circular markers. However, if the K-edge energy of an element falls between the high and low kVp average energies (e.g., tungsten at 69.5 keV) then the discontinuity at the K-edge energy now causes the attenuation at the low average energy to be less than the attenuation at the high average energy as shown by the two green circular markers. This inversion in attenuation values is what causes certain high-Z materials to be incorrectly placed entirely into the water equivalent image, thus leading to pseudo-cloaking of that material in the iodine equivalent image. The simulations also showed that pseudo-cloaking should occur on dual-energy spectral CT systems based on dual-source, fast kVp switching, and detector-based acquisition. The simulated range of pseudo-cloaking materials for the Philips IQon detector-based system used in this study spans from ytterbium (Z=70) to platinum (Z=78) as illustrated in FIG. 3.

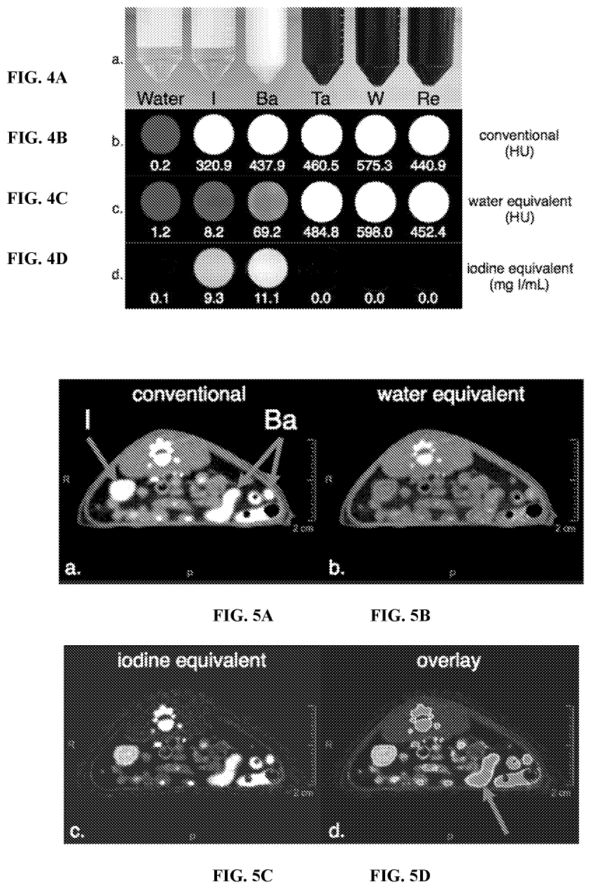

Contrast Agent Results. FIG. 4A shows a picture of six 50 mL plastic vials that contain the five contrast agents used in this study along with a vial of pure water. While the oral contrast agents were kept at their original concentrations (12 to 20 mg of element/mL) the iodine vial was diluted to from the stock concentration of 370 mg I/mL (i.e., Isovue-370) to 10 mg I/mL.

This dilution ensured that the iodine vial would have an attenuation value similar to the oral contrast vials while also representing typical iodine concentrations found in vivo. These vials were scanned together on a Philips IQon system using an Abdomen/Pelvis protocol (120 kVp, 182 mAs, field of view 256×256 mm, slice thickness 3 mm) to produce the conventional, water equivalent, and iodine equivalent axial images shown in FIGS. 4B, 4C, and 4D, respectively. FIGS. 4A to 4D also show the mean pixel values for each vial that were obtained using circular regions of interest. In the conventional CT image (FIG. 4B) each contrast agent is highly attenuating with mean values ranging from 321 HU (iodine) to 575 HU (tungsten). In the water equivalent image (FIG. 4C) the attenuation in the iodine and barium vials has been significantly reduced by 97% and 84%, respectively, while the tantalum, tungsten, and rhenium attenuation remains relatively unchanged (a slight increase of 3 to 5% is observed). However, in the iodine equivalent image (FIG. 4D) only the iodine and barium vials are visible while the tantalum, tungsten, and rhenium vials now have pixel values of 0 mg I/mL, showing that they are pseudo-cloaked in this image.

In vivo results. FIGS. 5A to 5D show axial images of the rat that was administered intravenous iodine and oral barium simultaneously. Each image is of the same axial slice but displayed using a different spectral CT method. In the conventional CT image (FIG. 5A) the iodine can be seen primarily in the kidneys (red arrow) and the barium can be seen in the bowel lumen (yellow arrows). In the water equivalent or virtual non-contrast image (FIG. 5B) both the iodine and barium have been removed leaving behind the soft tissues and bone (i.e, calcium). Since iodine and barium have such similar atomic numbers (and mass attenuation curves) they both appear in the iodine equivalent image or iodine map (FIG. 5C). As a result, any iodine within the bowel walls cannot be differentiated from the barium contained within the bowel lumen. This is emphasized in FIG. 5D (arrow) where the colorized iodine equivalent image has been overlaid onto the conventional image.

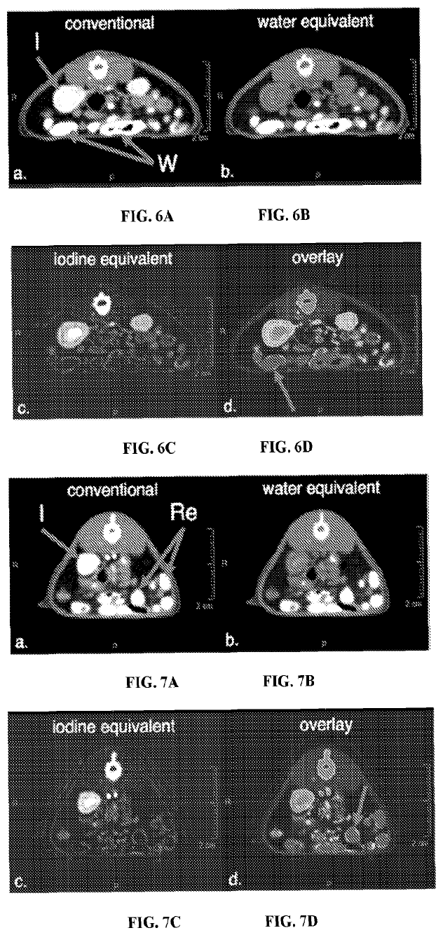

FIGS. 6A to 6D show similar axial spectral CT images of the rat that was administered intravenous iodine and oral tungsten simultaneously. In the conventional CT image (FIG. 6A) the iodine can be seen primarily in the kidneys (red arrow) and the tungsten can be seen in the bowel lumen (yellow arrows). In the water equivalent or virtual non-contrast image (FIG. 6B) only the iodine has been removed leaving behind the tungsten, soft tissues, and bone. In the iodine equivalent image or iodine map (FIG. 6C) only the iodine and calcium (i.e., bone) are seen while the tungsten is now completely pseudo-cloaked (i.e., 0 mg I/mL). When the iodine equivalent image is colorized and overlaid on top of the conventional image (FIG. 6D) the iodine in the bowel wall (shown as green pixels) can now be visually segmented from the tungsten in the bowel lumen (red arrow).

FIGS. 7A to 7D show similar axial spectral CT images for the rat that was administered intravenous iodine and oral rhenium simultaneously. It can be seen that the rhenium oral contrast agent behaves in the exact same way as the tungsten oral contrast agent from FIGS. 6A to 6D, where again pseudo-cloaking allows the iodine in the bowel wall (green pixels) to be visually segmented from the rhenium in the bowel lumen (FIG. 7D, red arrow). Similar preclinical pseudo-cloaking results were observed for the tantalum-based oral contrast agent (data not shown).

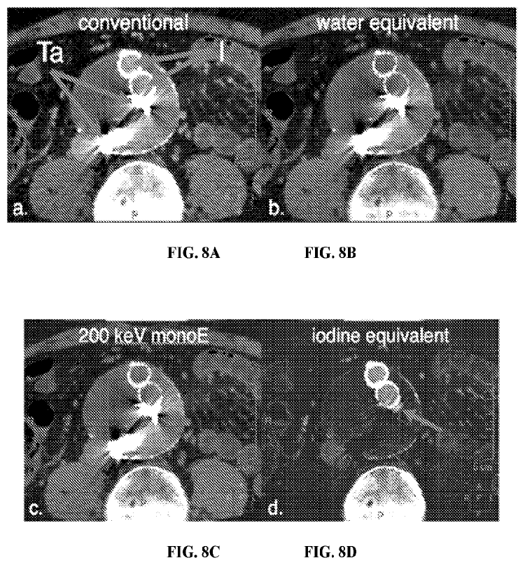

Clinical Results. FIG. 8A shows a conventional post-contrast CT image of a 71-year-old male patient with a large abdominal aneurysm. Within the aneurysm can be seen two metallic stents in the aortic bifurcation (I arrows) and a large amount of the tantalum containing Onyx liquid embolic agent (Ta arrows). It can also be seen that the streak artifacts caused by the highly attenuating Onyx agent, which measures over 2000 HU at its center, obscure the fine details within the aneurysm making it difficult to detect the presence of any endoleaks. FIG. 8B shows the water equivalent or virtual non-contrast image of the same slice where the iodine inside the aortic bifurcation has now been removed along with some calcium in the vertebrae, but the tantalum and its artifacts still remain. FIG. 8C shows that the 200 keV virtual mono-energetic (monoE) image, which is typically used to reduce streak artifacts caused by metal implants, has negligent effects on the tantalum. However, and importantly, in the iodine equivalent image or iodine map (FIG. 8D) the tantalum and any streak artifacts have now been completely removed by pseudo-cloaking to reveal a suspect type 2 endoleak within the aneurysm (arrow).

Due to their similar attenuation coefficients iodine and barium cannot be easily differentiated by x-ray CT, even when using spectral CT methods. This limits the diagnostic information obtained when using iodine-based and barium-based contrast agents simultaneously. However, it is shown herein that there is a certain range of high-Z elements that can be easily differentiated from iodine when imaged with the water and iodine two-material decomposition protocol. Due to a K-edge phenomenon associated with dual-energy spectral CT systems that the inventors call pseudo-cloaking, these high-Z elements are absent in the iodine equivalent image (i.e., have pixel values of ≤0 mg I/mL) and appear only in the water equivalent image. Therefore, these high-Z elements can be easily differentiated from iodine (or barium) by comparing the water equivalent image (i.e., the virtual non-contrast image) to the iodine equivalent image (i.e., the iodine map). These results agree with other examples of pseudo-cloaking of high-Z materials seen in the spectral CT literature (8,15,16).

In the present invention, tantalum, tungsten, and rhenium oral contrast images were taken using small animals on a clinical spectral CT system. Therefore, in order to improve the spatial resolution the inventors used a modified quality assurance (QA) protocol instead of a standard clinical abdominal/pelvis protocol. These pseudo-cloaking results translated to larger subjects imaged with standard clinical protocols, as evidenced by the Onyx embolic agent clinical data shown in FIGS. 8A to 8D. Pseudo-cloaking was demonstrated for three elements (tantalum, tungsten, and rhenium) on detector-based (Philips IQon) and dual-source (Siemens Force and Flash, phantom data not shown) spectral CT systems. Following the teachings of the present invention the skilled artisan can determine with minimal study and skill the exact range of the pseudo-cloaking elements for each dual-energy spectra CT system (i.e., dual-source, fast kVp switching, and detector-based) and how this range might depend upon settings such as kVp would be required for a complete understanding of the phenomenon. A barium-based oral contrast agents was used at it is approved by the FDA for x-ray CT.

These preclinical and clinical data show that pseudo-cloaking can be useful for the diagnosis of bowel ischemia (where the uptake of iodine in the bowel wall is reduced) and Crohn's disease (where the uptake of iodine in the bowel wall is increased or irregular). Likewise, the clinical data show that pseudo-cloaking can be useful for removing image artifacts caused by hyperattenuating materials that are based on pseudo-cloaking elements. Pseudo-cloaking could also be used to promote the development and FDA approval of new contrast agents for spectral CT.

As such, the present invention shows that certain high-Z elements appear pseudo-cloaked in iodine equivalent images derived from spectral CT water and iodine two-material decompositions. It was further found that pseudo-cloaking elements have pixel values of ≤0 mg iodine/mL in the iodine equivalent images. Using the present invention, simulations showed that pseudo-cloaking is due to a K-edge phenomena associated with dual-energy spectral CT systems. It was also found that pseudo-cloaking can be observed on both detector-based and dual-source spectral CT systems and fast kVp switching systems. Finally, pseudo-cloaking allows for the visual segmentation of iodine and certain high-Z elements.

Further, when used simultaneously with intravenous iodine contrast pseudo-cloaking of high-Z oral contrast agents can be used to image and diagnose bowel ischemia and Chron's disease with spectral CT. Moreover, it is demonstrated herein that pseudo-cloaking can be used to refine, improve, and/or develop new spectral CT contrast agents. The present inventors show that a certain high-Z elements that can be easily differentiated and visually segmented from iodine when imaged with the spectral CT water and iodine two-material decomposition protocol.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES—EXAMPLES 1 TO 7

1. Johnson, T. R., Dual-energy CT: general principles. AJR Am J Roentgenol, 2012. 199(5 Suppl): p. S3-8.
2. Goldman, L. W., Principles of CT and CT technology. J Nucl Med Technol, 2007. 35(3): p. 115-28; quiz 129-30.
3. Chandarana, H., et al., Iodine quantification with dual-energy CT: phantom study and preliminary experience with renal masses. AJR Am J Roentgenol, 2011. 196(6): p. W693-700.
4. Mongan, J., et al., In vivo differentiation of complementary contrast media at dual-energy CT. Radiology, 2012. 265(1): p. 267-72.
5. Rathnayake, S., et al., In vivo comparison of tantalum, tungsten, and bismuth enteric contrast agents to complement intravenous iodine for double-contrast dual-energy CT of the bowel. Contrast Media Mol Imaging, 2016.
6. Falt, T., et al., Material Decomposition in Dual-Energy Computed Tomography Separates High-Z Elements From Iodine, Identifying Potential Contrast Media Tailored for Dual Contrast Medium Examinations. J Comput Assist Tomogr, 2015. 39(6): p. 975-80.
7. Bolus, D. N., Dual-energy computed tomographic scanners: principles, comparisons, and contrasts. J Comput Assist Tomogr, 2013. 37(6): p. 944-7.
8. Liu, Y., et al., A high-performance ytterbium-based nanoparticulate contrast agent for in vivo X-ray computed tomography imaging. Angew Chem Int Ed Engl, 2012. 51(6): p. 1437-42.
9. Liu, Y., et al., Recent advances in ytterbium-based contrast agents for in vivo X-ray computed tomography imaging: promises and prospects. Contrast Media Mol Imaging, 2014. 9(1): p. 26-36.
10. Hinchcliffe, W. A., et al., Roentgenographic study of the human trachea with powdered tantalum. Radiology, 1970. 97(2): p. 327-30.
11. Nadel, J. A., et al., Powdered tantalum. N Engl J Med, 1970. 283(6): p. 281-6.
12. Tones, A. S., et al., Biological performance of a size-fractionated core-shell tantalum oxide nanoparticle x-ray contrast agent. Invest Radiol, 2012. 47(10): p. 578-87.
13. Yu, S. B. and A. D. Watson, Metal-Based X-ray Contrast Media. Chem Rev, 1999. 99(9): p. 2353-78.
14. Yu, S. B., et al., Dimeric W3SO3 cluster complexes: synthesis, characterization, and potential applications as X-ray contrast agents. Inorg Chem, 2001. 40(7): p. 1576-81.
15. Yu, S., et al., Cuboidal W3S4 cluster complexes as new generation X-ray contrast agents. Inorg Chem, 2000. 39(6): p. 1325-8.
16. Chou, S. W., et al., In vitro and in vivo studies of FePt nanoparticles for dual modal CT/MRI molecular imaging. J Am Chem Soc, 2010. 132(38): p. 13270-8.

### REFERENCES—EXAMPLE 8

1. Johnson T R, Krauss B, Sedlmair M, Grasruck M, Bruder H, Morhard D, Fink C, Weckbach S, Lenhard M, Schmidt B, Flohr T, Reiser M F, Becker C R. Material differentiation by dual energy CT: initial experience. European radiology 2007; 17(6):1510-1517.
2. McCollough C H, Leng S, Yu L, Fletcher J G. Dual- and Multi-Energy CT: Principles, Technical Approaches, and Clinical Applications. Radiology 2015; 276(3):637-653.
3. Tamm E P, Le O, Liu X M, Layman R R, Cody D D, Bhosale P R. “How to” incorporate dual-energy imaging into a high volume abdominal imaging practice. Abdom Radiol 2017; 42(3):688-701.
4. Mileto A, Sofue K, Marin D. Imaging the renal lesion with dual-energy multidetector CT and multi-energy applications in clinical practice: what can it truly do for you? European radiology 2016.
5. Mileto A, Marin D, Alfaro-Cordoba M, Ramirez-Giraldo J C, Eusemann C D, Scribano E, Blandino A, Mazziotti S, Ascenti G. Iodine quantification to distinguish clear cell from papillary renal cell carcinoma at dual-energy multidetector CT: a multireader diagnostic performance study. Radiology 2014; 273(3):813-820.
6. Hubbell J H. Photon Mass Attenuation and Energy-Absorption Coefficients from 1 Key to 20 Mev. Int J Appl Radiat Is 1982; 33(11):1269-1290.
7. Rabin O, Perez J M, Grimm J, Wojtkiewicz G, Weissleder R. An X-ray computed tomography imaging agent based on long-circulating bismuth sulphide nanoparticles. Nat Mater 2006; 5(2):118-122.
8. Mongan J, Rathnayake S, Fu Y J, Wang R T, Jones E F, Gao D W, Yeh B M. In Vivo Differentiation of Complementary Contrast Media at Dual-Energy CT. Radiology 2012; 265(1):267-272.
9. Seltzer S M. Calculation of Photon Mass Energy-Transfer and Mass Energy-Absorption Coefficients. Radiat Res 1993; 136(2):147-170.
10. Heismann B, Balda M. Quantitative image-based spectral reconstruction for computed tomography. Med Phys 2009; 36(10):4471-4485.
11. Alvarez R E, Macovski A. Energy-Selective Reconstructions in X-Ray Computerized Tomography. Phys Med Biol 1976; 21(5):733-744.
12. Brooks R A, Dichiro G. Split-Detector Computed Tomography—Preliminary-Report. Radiology 1978; 126(1):255-257.
13. Brooks R A. Quantitative Theory of Hounsfield Unit and Its Application to Dual Energy Scanning. Journal of computer assisted tomography 1977; 1(4):487-493.
14. Hounsfield G N. Computerized transverse axial scanning (tomography). 1. Description of system. The British journal of radiology 1973; 46(552):1016-1022.
15. Rathnayake S, Mongan J, Torres A S, Colborn R, Gao D W, Yeh B M, Fu Y. In vivo comparison of tantalum, tungsten, and bismuth enteric contrast agents to complement intravenous iodine for double-contrast dual-energy CT of the bowel. Contrast Media Mol Imaging 2016; 11(4):254-261.
16. Falt T, Soderberg M, Wasselius J, Leander P. Material Decomposition in Dual-Energy Computed Tomography Separates High-Z Elements From Iodine, Identifying Potential Contrast Media Tailored for Dual Contrast Medium Examinations. Journal of computer assisted tomography 2015; 39(6):975-980.
17. Yeh B M, FitzGerald P F, Edic P M, Lambert J W, Colborn R E, Marino M E, Evans P M, Roberts J C, Wang Z J, Wong M J, Bonitatibus P J, Jr. Opportunities for new CT contrast agents to maximize the diagnostic potential of emerging spectral CT technologies. Adv Drug Deliv Rev 2016.

1. An enteric contrast agent formulation comprising:
an enteric contrast medium comprising particles comprising atoms of an element with an atomic number from 70 to 77; and
a pharmaceutically acceptable vehicle in which the particles are dispersed.

2. The contrast agent of claim 1, wherein the element is invisible or cloaked in an iodine spectral computer tomography image.

3. The contrast agent of claim 1, wherein the element is in a compound selected from Rhenium(VII) sulfide (Re₂S₇), or a non-soluble Tungstate (X—WO₄).

4. The contrast agent of claim 1, wherein the element is non-absorbable in the gut.

5. The contrast agent of claim 1, wherein the element is non-radioactive.

6. The contrast agent of claim 1, wherein the element is selected from Yb, Lu, Hf, Ta, W, Re, Os, or Ir.

7. The contrast agent of claim 1, wherein the particles are coated with at least one of: a viscosity modifier and water retention agent to form a colloidal nanoparticle that is pseudo-cloaking; an enteric coating; a material compatible with enteric administration of the formulation; adapted for oral administration; or the coating comprises a water-soluble polymer.

8. (canceled)

9. (canceled)

10. The contrast agent of claim 1, wherein the atoms are defined further as comprising particles of a material selected from microparticles and nanoparticles, wherein the particles comprise atoms of an element with an atomic number from 70 to 77.

11. The contrast agent of claim 1, wherein the enteric contrast medium is defined further as comprising a first subpopulation of atoms having a first atomic number and a second subpopulation of atoms having a second atomic number, wherein the first atomic number and the second atomic number are different atomic numbers.

12. (canceled)

13. The contrast agent of claim 1, wherein the particle is essentially water insoluble or slightly water soluble.

14. The contrast agent of claim 1, wherein the element further comprises one or more atoms selected from oxygen and sulfur forming a compound with the element or the element is in the form of an oxide, a carbonate, a borate, a hydroxide, a phosphate, and a salt of an organic acid of the element.

15. (canceled)

16. The contrast agent of claim 1, wherein the formulation is in a form selected from a suspension, a colloid, an emulsion, a hydrogel, or a combination thereof.

17. The contrast agent of claim 1, wherein from about 10% (w/w) to about 90% (w/w) of the weight of the formulation is the contrast material particles, or about 30% (w/w) to about 70% (w/w) of the weight of the formulation is the contrast material particles.

18. (canceled)

19. The contrast agent of claim 1, wherein the coating comprises a polymer which is a member selected from a poly(alkylene oxide), a poly(amino acid), a poly(ester) polymer, a polysaccharide, polyvinylpyrrolidone, a polyvinyl) polymer, a poly(ethylene imine) polymer, a poly(acrylic) polymer, a poly(siloxane) polymer, a protein, a dendrimer and a combination thereof; the coating comprises an organic molecule with a molecular weight of less than about 3,000 Daltons; or the coating comprises an organic molecule with a molecular weight of less than about 3,000 Daltons, which is a member selected from an organic acid, alcohol, amine, an oligosaccharide and their derivatives and analogs (e.g. perfluoroalkyl chain, fluoroalkyl chain) and a combination thereof.

20. (canceled)

21. (canceled)

22. The contrast agent of claim 1, wherein the size of the particles is from: about 1 nm to about 500 microns, less than about 200 nm, from about 200 nm to about 5 microns, about 1 micron to about 50 microns, or greater than about 50 microns.

23. The contrast agent of claim 1, wherein the pharmaceutically acceptable vehicle comprises an aqueous medium, further comprising an additive to retard dehydration of the formulation in the bowel, a flavoring agent, a thickening agent, a suspending agent, a flow agent, a pH buffer and a combination thereof.

24. The contrast agent of claim 1, wherein the element has a Z<83 (Bismuth).

25. A method of making a colloidal nanoparticle contrast agent for enhanced spectral CT image of a subject comprising:
suspending a nanopowder comprising an element selected from Z=70 (Ytterbium) to Z=78 (Platinum) in water; and
coating the nanopowder with a sufficient amount of a viscosity modifier and water retention agent to form a colloidal nanoparticle that is pseudo-cloaking.

26. The method of claim 25, further comprising the step of adding one or more additives, stabilizers, adhesives, flavorants, or preservatives or.

27. The method of claim 25, further comprising the step of adding one or more additives, stabilizers, adhesives, flavorants, or preservatives selected from at least one of: bentonite, dimethylpolysiloxane 200, dimethylpolysiloxane 1000, D-sorbitol, D-mannitol, saccharin sodium salt hydrate, sodium benzoate, or sodium citrate.

28. The method of claim 25, wherein the nanopowder is selected from at least one of Yb, Lu, Hf, Ta, W, Re, Os, Ir, tantalum oxide, tungsten carbide, tungsten trioxide, sodium tungstate, or rhenium sulfide.

29. The method of claim 25, wherein the step of coating the nanoparticle comprises at least one of sonicating or stirred for an amount of time sufficient to obtain the colloidal nanoparticles or homogenous colloidal nanoparticles.

30. (canceled)

31. The method of claim 25, wherein the element has a Z<83 (Bismuth).

32. A method of acquiring a contrast enhanced spectral CT image of a subject comprising:
administering to the subject a diagnostically effective amount of an enteric contrast medium formulation comprising particles of an element having an atomic number from 70 to 77 dispersed in a pharmaceutically acceptable vehicle; and
acquiring the image of the subject.

33. (canceled)

34. The method of claim 32, wherein the image is an image of a region selected from the abdomen and pelvis of the subject.

35. The method of claim 32, wherein the method further comprises administering to the subject a second contrast medium different from the enteric contrast medium, and the second contrast medium is administered through a route selected from oral administration, intrathecal administration, intravesicular administration, enteric administration, anal administration and intravascular administration.

36. The method of claim 32, wherein the enteric contrast medium and the second contrast medium are distinguishable from each other in the image.

37. The method of claim 32, wherein the enteric contrast medium does not appear in an IodineNoWater image.

38. The method of claim 32, wherein the enteric contrast medium remains in the WaterNoIodine image and is enhanced when compared to a conventional CT image.

39. The method of claim 32, wherein an image of a bowel can be segment by performing a pixel wise comparison of the WaterNoIodine and conventional CT images.

40. The method of claim 32, wherein the particles are coated with a viscosity modifier and water retention agent to form a colloidal nanoparticle that is pseudo-cloaking.

41. The method of claim 32, wherein the enteric contrast agent is administered to the subject by delivery through:
a natural cavity selected from the mouth, vagina, bladder, rectum and urethra;
a surgically created space selected from an ileal pouch, and a neobladder; or
medical device selected from a catheter, a tube, a reservoir, a pouch and a pump.

42. A kit comprising:
a first vial containing the enteric contrast medium a diagnostically effective amount of the enteric contrast medium formulation comprising an atomic number from 70 to 77 dispersed in a pharmaceutically acceptable vehicle; and
directions for formulating the enteric contrast medium, the second contrast medium or a combination thereof.

43. The kit of claim 42, further comprising a second vial containing a second contrast medium for intravenous administration.
